(19) United States
(12) Patent Application Publication
(10) Pub. No.: US 2001/0037340 A1
(43) Pub. Date: Nov. 1, 2001

(54) System and method for providing medical services and administering medical protocols to individuals based upon biological testing

(76) Inventors: Daniel Hawkins, Bethesda, MD (US); Douglas Nunes, Potomac, MD (US); Alex Titomirov, Great Falls, VA (US)

Correspondence Address: Roberts Abokhair & Mardula, LLC, 11800 Sunrise Drive, Suite 1000, Reston, VA, 20191-5302, US

(21) Appl. No.: 09/773,700

(22) Filed: Feb. 1, 2001

Related U.S. Application Data
(63) Continuation-in-part of application No. 09/498,804, filed on Feb. 7, 2000.
(63) Continuation-in-part of application No. 09/645,696, filed on Aug. 25, 2000.

(51) Int. Cl. G06F 17/30; A61B 5/00

(52) U.S. Cl. 707/104.1; 705/2; 707/10; 600/300

(57) ABSTRACT

A system and method for providing medical intervention services to individuals based upon a biological sample being provided to an analysis processor, and the analysis processor sending information to the individual and queries to sources of good and services. The analysis processor automatically formats queries on behalf of the individual based upon any conditions assessed. Thereafter, the sources of medical intervention services can send information and offers of goods preferably through the analysis processor to the individual, thereby maintaining the anonymity of the individual. If the individual wished, offers and information can be sent directly to the individual.

### RELATIONSHIP TO PRIOR INVENTIONS

This application is a continuation-in-part of application Ser. No. 09/498,804, filed Feb. 7, 2000 and application Ser. No. 09/645,696, filed Aug. 25, 2000, from which priority is claimed

### FIELD OF THE INVENTION

This invention relates generally to providing medical intervention services to patients. More particularly the present invention is a system and method for noting a particular user's condition and providing advise on medical intervention services key to that condition.

### BACKGROUND OF THE INVENTION

As the population of the United States to continues to age, this older age group will begin to exhibit symptoms of various diseases and conditions (collectively “conditions”) which require medical care. Simultaneously, the technology of disease assessment and prediction of certain conditions continues to improve as the science of analyzing proteins, small molecules, DNA, and a host of other genetic tests begins to mature.

Mapping of the human genome has been in process by both the government and private industry for the past ten years. At the same time new research has lead to specific tests for conditions that are evidenced by genes and certain gene expressions. Further the methods for the detecting of genomic, proteomic and other biological factors have dramatically improved.

Similarly, genetic engineering and research is extremely active in the agricultural marketplace where both plants and animals are the subject of genetic engineering and commercialization.

Research is rapidly progressing on DNA chips from, for example, Affymetrix, and RNA and protein chips available from, for example, Ciphergen which will allow genes, their expression and proteins to be identified on a particular chip. Further Micro Arrays such as those of Nycomed Amersham/Amersham Pharmacia Biotec allow gene detection via very small detectors, thereby reducing the size of the overall detector package. Further, advances in fiber optic probes for example by Illumina, Inc. now allow 250,000 discrete sensors to fit on a probe the diameter of a pin head for proteomic studies. For purposes of this application, such chips, arrays and probes are referred generally as “Analysis Devices.” Other similar technologies are available from PE Biosystems, Nycomed Amersham, Nanogen, Hyseq, Caliper, Clinical Micro Sensors. Specifically, Nanogen possesses electronic concentration and hybridization technology that is suited to the present invention. Such technology is embodied in a disposable cartridge comprising a semiconductor microchip and electrical and fluidic connections for use in analysis equipment. The following U.S. patents discuss this technology, the contents of the below listed patents are incorporated herein by reference in their entirety:

U.S. Pat. No. 6,099,803 “Advanced active electronic devices for molecular biological analysis and diagnostics”

U.S. Pat. No. 6,071,394 “Channel-less separation of bioparticles on a bioelectronic chip by dielectrophoresis ”

U.S. Pat. No. 6,068,818 “Multicomponent devices for molecular biological analysis and diagnostics”

U.S. Pat. No. 6,051,380 “Methods and Procedures for Molecular Biological Analysis and Diagnostics”

U.S. Pat. No. 6,048,690 “Methods for Electronic Fluorescent Perturbation for Analysis and Electronic Perturbation Catalysis for Synthesis ”

U.S. Pat. No. 5,849,486 “Methods For Hybridization Analysis Utilizing Electronically Controlled Hybridization”

U.S. Pat. No. 5,605,662 “Active Programmable Electronic Devices for Molecular Biological Analysis and Diagnostics”

Other analysis techniques also exist and are suitable for use with the present inventions. For example, U.S. Pat. No. 6,045,676 was issues to Mathies et al. for a “Electrochemical detector integrated on microfabricated capillary electrophoreses chips” for use in detecting molecules. The contents of this patent are incorporated herein by reference in its entirety.

The LabCD manufactured by Gamera Bioscience Corp. also represents technology that is suitable to sample preparation that would find use in the present invention. The capabilities and format of the LabCD are described in U.S. Pat. No. 6,063,589 which is incorporated herein by reference in its entirety. These analysis devices can then be used to determine if the particular gene in question is working properly with other genes, or if other biological factors are present which may be indicative of certain medical conditions.

The trend therefore is to continue to map human genes and to develop understanding regarding how they are expressed in a body or in body fluids (for example, blood, urine, saliva, sperm, sweat and milk). For example it would be very useful for certain individuals to obtain their own gene expression profile so that it can be used for assessing their normal versus abnormal condition states. If, for example, an individual knows the difference between the normal expression of genes and the deviations from normal which are statistically important, that could prevent for example, breast cancer, colon cancer, and other conditions that are reflected by a mutated gene. It would also be useful to have the individual's gene expression profile, which would be a baseline or standard, for subsequent comparison of normal versus abnormal conditions.

This type of testing will lead to a prediction of conditions well before the manifestation of any symptoms occurs. Further, individual persons will begin to rely upon such advanced proteomic and genetic testing in order to assess the potential for those individuals manifesting a particular condition. In such cases persons will want to be more pro-active in there health care and subsequent treatment.

As part of the notion of being pro active in healthcare and subsequent treatment, it is important that individuals who have been diagnosed with a medical condition or the potential of a medical condition from the genomic or proteomic testing above that such individuals have information of the availability and types of medical interventions and procedures (collectively “medical events”) that will potentially be required to deal with such conditions. Further, before a user decides to seek out specific practitioners who deal with the various events that may be required, it would also be useful for individuals to have information on the type and frequency of various medical events so that the individual can select the time to begin such interventions and, more importantly, to be reminded of the need for the various interventions selected.

### SUMMARY OF THE INVENTION

It is yet another objective of the present invention to use any condition information generated from a biological sample to search a data base and apprize a user of medical procedures and treatment options that are keyed to the condition detected.

It is still another objective of the present invention to advise a user of sources of services that are available that are keyed to a particular condition.

It is still another objective of the present invention to independently provide alerts to those sources of services so that organization information from those sources can be provided to the individual having the condition or potential for the condition detected.

It is a further objective of the present invention to advise a user of the types of intervention that are available that are keyed to the particular condition that is assessed.

It is still another objective of the present invention to advise a user of sources for the treatment or interventions which are suggested and which are keyed to the condition detected.

It is still another objective of the present invention to provide periodic notification to an individual of the need for interventions keyed to the condition detected.

These and other objectives of the present invention will become apparent to those skilled in the art from a review of the specification that follows.

The present invention is a system and method for providing automated searching to users who are determined to have or potentially may develop a particular condition. Initially, a user provides a biological sample for testing. This sample can be obtained in a variety of ways such as those indicated in patent application Ser. No. 09/498,804, filed Feb. 7, 2000, and application Ser. No. 09/645,696, filed Aug. 25, 2000, which are incorporated herein by reference in their entirety.

A user provides the appropriate biological sample and for example, a proteomic analysis is conducted. The biological sample is provided on a chip, centripetal analysis device, or any other type of analysis device. Information is read from the analysis device and, preferably, information is provided in a secure fashion over the Internet to an analysis server. The analysis server determines the likelihood of any particular condition and provides that information to the user. In addition, the system of the present invention also determines the need for a particular type of genetic test based upon the profile determined. All user files are strictly confidential.

When the user first registers with the system of the present invention to perform such analysis, the system of the present invention will ask if the user desires to be notified of the sources of medical intervention services or wishes simply to have the sources of such services identified to the user. The identification of the user is always kept confidential and the user can then choose to contact any sources of medical intervention services that may be identified. If the user desires, identification information can be provided to individual sources of medical intervention services that relate to a particular condition. Those sources can then provide information to the user.

As part of any business model of the present invention, such sources must agree to keep the identification of the individual strictly confidential as well. In this fashion the user can have some assurance that his or her name will not be given out to others except with express permission.

Once the particular condition of the user is assessed, the analysis processor of the present invention will perform an in depth search noting various providers of medical intervention services and protocols which are germane to the condition which was assessed. Thereafter identification of sources medical intervention services can be provided to the user for subsequent purchase. In addition, key information on the condition that has been assessed by the analysis processor is provided to the user so that the user can be informed of the particular condition detected.

Once particular sources of medical intervention services have been identified for the user, the user can also opt to have alerts sent to the various sources of noting either anonymously or with direct contact information that the system of the present invention has a user with a particular condition or the potential therefore. This “opting in” allows a user to maintain the user's privacy by not having such alerts sent unless specifically requested. However, if the user does request such alerts to be sent, the user has saved substantial time and effort by directing the system of the present invention to send such alerts to various sources of medical intervention services. These sources of medical intervention services can then provide information to those users where a particular condition has been assessed.

Security for the user is provided either by a contractual obligation of the various products sources to keep such information confidential or, the system of the present invention can simply assign a random identifier to the particular user, and all offers can be sent to the server of the present invention which will then forward that information to the user thereby maintaining the user's security.

### BRIEF DESCRIPTION OF THE DRAWINGS

FIG. 1 illustrates the flow of providing information to the individual user.

FIG. 2 illustrates the flow of providing offers to the individual user.

FIG. 3 illustrates an overview architecture according to one embodiment of the present invention.

FIG. 4 illustrates an overview architecture of the present invention according to one embodiment whereby offers are provided directly to the user.

### DETAILED DESCRIPTION OF THE INVENTION

The present invention is a system and method for assessing the presence or the potential for a particular condition based upon a biological sample and subsequently advising a user of sources of medical intervention services that are keyed to the particular condition that is assessed. For example, and without limitation, genetic testing may indicate that a person has a higher than average chance of contracting colon cancer. In this instance, medical service providers offering colonoscopy

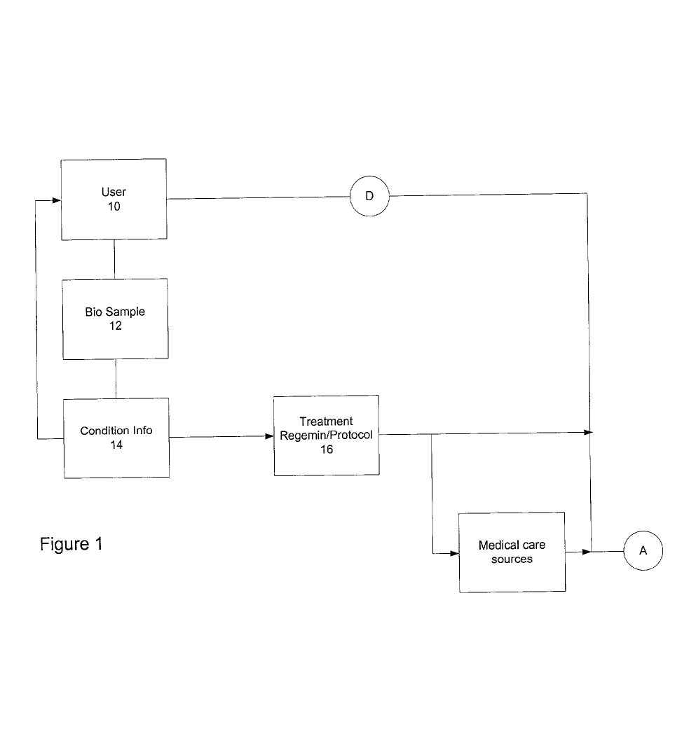

Referring first to FIG. 1 the overall information flow of the present invention is illustrated. User 10 offers a biological sample 12 to be used for the assessment of a particular fiscal condition or to simply do an assessment for the presence of a variety of physical conditions. That biological sample 12 is analyzed by an analysis processor and test results and condition information 14 is generated. This information is fed back to the user 10 so that the user can have the most up to date information concerning the condition that has been assessed.

The analysis server of the present invention (as will be discussed later) uses an internal health related browser to construct queries over, preferably, the Internet to retrieve particular products 16, medications 18, food products 20 that relate to the particular condition information 14 that was assessed for the user 10. Once the products 16, medications 18, and food products 20 have been identified, the analysis processor then searches for various sources, 22, 24, and 26 for the products 16, medications 18, food products 20 that have been identified. For example, there can be many sources of insulin or diabetes related products. These multiple sources are researched, identified, and provided back to the user 10 for subsequent action.

Thereafter user 10 is free to contact the various sources 22, 24, and 26 to determine what offers for medical intervention services might be available.

However, it can well be that given the large number of sources and the priority on the user's time, that contacting a wide variety of sources may be too time consuming or out of the question. Where this is the case, a particular user may miss out on offers or further information on products that may be of assistance. To combat this particular difficulty, the system of the present invention can allow the user to “opt in” to an alert service whereby sources for products, medications and food products are alerted to the fact that a particular user needs that information.

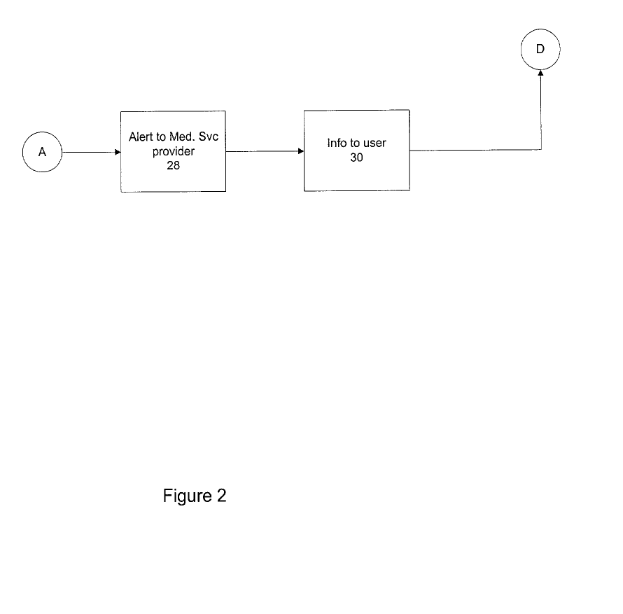

Referring to FIG. 2 the process flow for obtaining information continues. Once a source for a particular product has been identified, and assuming a user has opted into the alert service, an alert is provided by the analysis processor of the present invention to the source. Thereafter, the source can prepare an offer to the user and convey that offer to the user. Thus an alert to the source of products 38 is made and the product source can relay an offer 30 to the user. Similarly an alert to the source for medication 32 can be made and an offer made to the user relating to those medications 34. Finally an alert to the source of food products 36 can be provided and an offer made by that source to the user 38 can be generated.

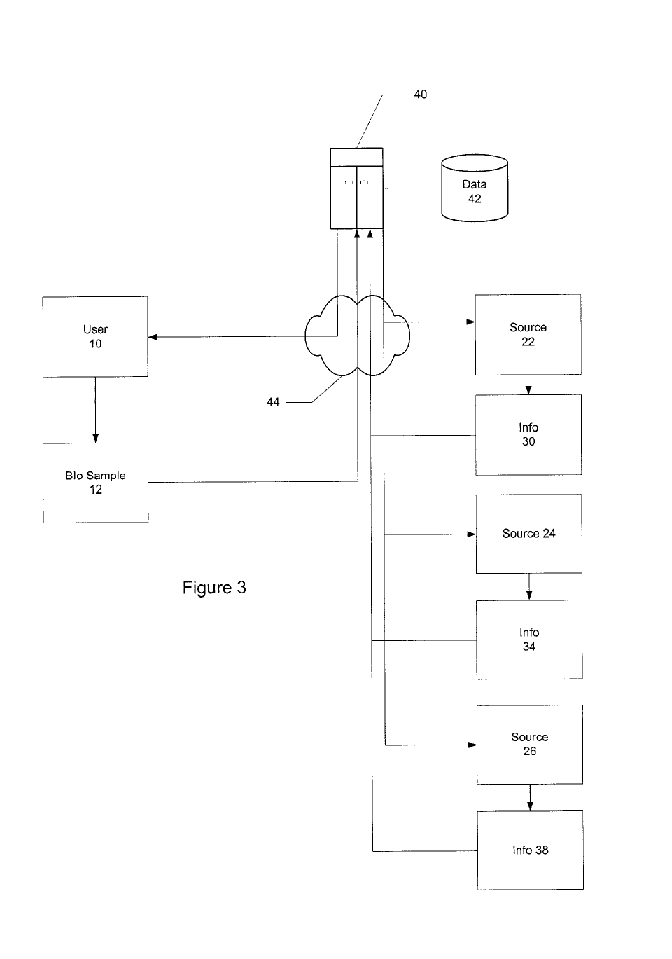

However interested the user may be in particular alerts, the user may also have a privacy and security concern that is paramount for the particular user. Thus although the user may wish to have information on products and services, the user may not wish his or her name to be identified to organizations outside the analysis server. Where this is the case and referring to FIG. 3, the secure providing of offer information is illustrated. User 10 provides biological sample 12 over network 44 which is preferably but without limitation, the Internet to the analysis processor 40. Analysis processor 40 analyses the biological sample and determines what if any condition exists or has the potential for existing and provides that information back to user 10. In doing this assessment, analysis processor 40 queries its own database 42 of biological information to make the particular assessment of a condition. In addition, however analysis processor 40 creates a query using its health related browser which provides an alert to source 28 which has a product for services which is germane to the particular user. That source in turn creates an offer 30 which is provided back over the network 44 to the analysis processor. Thereafter the analysis processor which has identified the user only by means of an anonymous user identifier receives the information, notes the user identifier number, and associates it with a particular user. The information is then sent directly to user 10. Similarly any other alerts to additional sources 32 and offers 34 are presented in the same anonymous fashion via the analysis server to the user 10. In this fashion the anonymity of the user 10 is maintained.

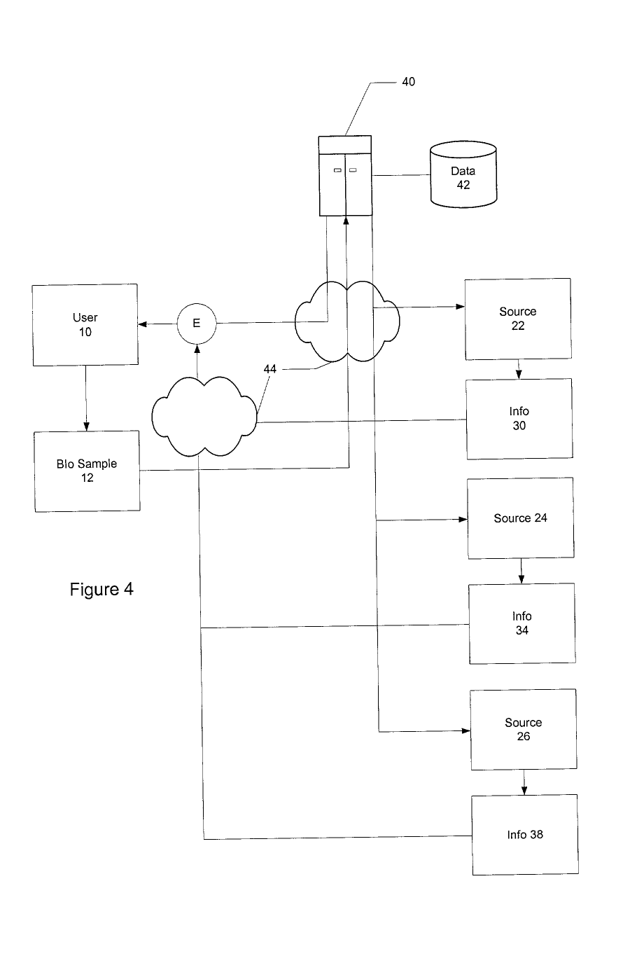

Referring to FIG. 4, direct response of sources to the user is illustrated. Again the user 10 provides a biological sample 12 to analysis processor 40 over the internet 44. The analysis processor searches its own database 42 for information and returns that condition information to the user 10 over the internet 44. In this instance however, the user 10 is given the option of directly receiving offers from sources relating to the condition that is assessed by analysis processor 40. IF the user “opt-in” to such a notice program, the analysis processor 40 will, upon assessing the presence of the potential for, a particular condition, automatically generate a query to all sources of products 22, medications 24, and food sources 26 that the user wishes to receive offers for medical intervention services relating to the condition assessed. Thereafter, the respective sources create offers, 30, 34, and 38 which are sent directly to the user 10 over the internet 44.

A system and method of providing information relating to the presence of a medical condition based upon biological testing has now been illustrated. It will be appreciated by those skilled in the art that other variation of the present invention are possible without departing from the scope of the invention as disclosed. For example, while the internet has been noted as the medium of transmission, other networks will also suffice for the present invention such as other types of wired and wireless network. Such networks will suffice so long as they are capable of transmitting information to the user. Thus the internet use is not meant as a limitation.

We claim:

1. A method for providing medical intervention services information to individuals based upon biological testing comprising:
an individual submitting a biological sample to an analysis processor over a network;
the analysis processor assessing the biological sample for a condition;
the analysis processor retrieving information from a database on the condition and providing the information to the individual over the network;
the analysis processor automatically formatting a query to retrieve information from sources of medical intervention services relating to the condition assessed; and
the sources of medical intervention service transmitting information relating to the condition to the individual over the network.

2. The method of claim 1 further comprising:
the analysis processor's automatically formatting a query further comprises the analysis processor assigning a random alias to the individual and transmitting that random alias along with the automatically generated query to the sources of medical intervention services; and
wherein the sources of medical intervention services transmitting information relating to the condition to the individual further comprises the sources providing the information together with the random alias to the analysis processor and the analysis processor associating the random alias with the individual and the analysis processor transmitting the information from the sources to the individual identified.

3. The method for providing medical intervention services information to individuals based upon biological testing of claim 1 wherein the biological sample comprises proteomic information.

4. The method for providing medical intervention services information to individuals based upon biological testing of claim 1 wherein the biological sample comprises genomic information.

5. The method for providing medical intervention services information to individuals based upon biological testing of claim 1 wherein the biological information is genomic information and is taken from the group consisting of DNA and RNA.

6. The method for providing medical intervention services information to individuals based upon biological testing of claim 1 wherein the biological sample comprises information concerning sugars in the biological sample.

7. The method for providing medical intervention services information to individuals based upon biological testing of claim 1 wherein the network is the Internet.

8. The method for providing medical intervention services information to individuals based upon biological testing of claim 1 wherein the network is a wireless network.

9. The method for providing medical intervention services information to individuals based upon biological testing of claim 1 wherein the sources of medical intervention services relating to the condition assessed comprises physicians.

10. The method for providing medical intervention services information to individuals based upon biological testing of claim 1 wherein the sources of medical intervention services relating to the condition assessed comprises treatment centers.

11. The method for providing medical intervention services information to individuals based upon biological testing of claim 1 further comprising reminding the individual on a periodic basis of the need to perform intervention tasks relating to the condition assessed.

12. A system for providing medical intervention services information to individuals based upon biological testing comprising:
an analysis device for receiving a biological sample from and individual and for transmitting the biological sample to an analysis processor over a network;
the analysis processor connected to the network further comprising instructions for assessing the biological sample for a condition;
the analysis processor further comprising instructions for retrieving information from a database on the condition and for providing the information to the individual over the network;
the analysis processor further comprising instructions for automatically formatting a query to retrieve information from sources of medical intervention services connected to the network, the information relating to the condition assessed; and
whereby the sources of medical intervention services transmit information relating to the condition to the individual over the network.

13. The system for providing medical intervention services information to individuals based upon biological testing of claim 12 wherein the analysis processor further comprises instructions for assigning a random alias to the individual and for transmitting that random alias along with the automatically generated query to the sources of medical intervention services; and
wherein the sources of medical intervention services comprise instructions for providing the information together with the random alias to the analysis processor; and
wherein the analysis processor further comprises instructions for associating the random alias with the individual and for transmitting the information from the sources to the individual identified.

14. The system for providing medical intervention services information to individuals based upon biological testing of claim 12 wherein the biological sample comprises proteomic information.

15. The system for providing medical intervention services information to individuals based upon biological testing of claim 12 wherein the biological sample comprises genomic information.

16. The system for providing medical intervention services information to individuals based upon biological testing of claim 12 wherein the biological information is genomic information and is taken from the group consisting of DNA and RNA.

17. The system for providing medical intervention services information to individuals based upon biological testing of claim 12 wherein the biological sample comprises information concerning sugars in the biological sample.

18. The system for providing medical intervention services information to individuals based upon biological testing of claim 12 wherein the network is the Internet.

19. The system for providing medical intervention services information to individuals based upon biological testing of claim 12 wherein the network is a wireless network.

20. The system for providing medical intervention services information to individuals based upon biological testing of claim 12 wherein the sources of medical intervention services relating to the condition assessed comprises sources of devices.

21. The system for providing medical intervention services information to individuals based upon biological testing of claim 12 wherein the sources of medical intervention services relating to the condition assessed comprises sources of pharmaceutical products and information.

22. The system for providing medical intervention services information to individuals based upon biological testing of claim 12 wherein the sources of medical intervention services relating to the condition assessed comprises sources of food products.
